(19) United States
(12) Patent Application Publication
(10) Pub. No.: US 2024/0130681 A1
(43) Pub. Date: Apr. 25, 2024

(54) ELECTRODE PLACEMENT CALIBRATION

(71) Applicant: Meta Platforms Technologies, LLC, Menlo Park, CA (US)

(72) Inventors: Christopher William James Rittaler, Marysville, WA (US); Filipp Demenschonok, Bothell, WA (US)

(21) Appl. No.: 18/488,989

(22) Filed: Oct. 16, 2023

Related U.S. Application Data
(60) Provisional application No. 63/417,934, filed on Oct. 20, 2022.

(51) Int. Cl. A61B 5/00; A61B 5/318; A61B 5/369; G06F 3/01

(52) U.S. Cl. CPC A61B 5/684; A61B 5/0048; A61B 5/318; A61B 5/369; A61B 5/7246; A61B 5/7278; A61B 5/742; G06F 3/015; A61B 5/6803; A61B 5/6814; A61B 5/6824; A61B 2560/0238; A61B 2560/0406; A61B 2560/045; A61B 2562/046

(57) ABSTRACT

The disclosed method may include driving a reference signal into a body and detecting, in response to the reference signal, a plurality of biosignal measurements using at least one electrode of a biosensing device. The method may further include determining, based on the plurality of biosignal measurements, a relative location of the at least one electrode with respect to the body, and providing feedback based on the relative location of the at least one electrode. Various other methods, systems, and computer-readable media are also disclosed.

### CROSS REFERENCE TO RELATED APPLICATION

This application claims the benefit of U.S. Provisional Application No. 63/417,934, filed 20 Oct. 2022, the disclosure of which is incorporated, in its entirety, by this reference.

### BRIEF DESCRIPTION OF THE DRAWINGS

The accompanying drawings illustrate a number of exemplary embodiments and are a part of the specification. Together with the following description, these drawings demonstrate and explain various principles of the present disclosure.

FIGS. 1A and 1B are illustrations of example applications of a reference stimulus.

FIG. 2 is a flow diagram of an exemplary method for electrode placement calibration.

FIG. 3 is an example graph of measuring a signal in response to a reference stimulus.

FIG. 4 is an illustration of exemplary augmented-reality glasses that may be used in connection with embodiments of this disclosure.

FIG. 5 is an illustration of an exemplary virtual-reality headset that may be used in connection with embodiments of this disclosure.

FIGS. 6A and 6B are illustrations of an exemplary human-machine interface configured to be worn around a user's lower arm or wrist.

FIGS. 7A and 7B are illustrations of an exemplary schematic diagram with internal components of a wearable system.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the present disclosure covers all modifications, equivalents, and alternatives falling within the scope of the appended claims.

### DETAILED DESCRIPTION OF EXEMPLARY EMBODIMENTS

Electromyography (EMG) devices and other biosensing or biosignal measurement devices (e.g., electrocardiograms (ECG), electroencephalograms (EEG), etc.) may measure electrical activity of a human body. Biosensing devices may measure EMG and other electrical signals emitted by the human body to measure certain aspects of the body, such as in a medical diagnostic context. These electrical signals may also be used as inputs for input devices of computing devices. These electrical signals are often small, for example in an order of magnitude of micro-volts.

Due to the precision necessary for accurate measurements, a placement of electrodes of a biosensing device along a body may greatly influence accuracy of biosignals. For example, physiological features, such as skin near a bone structure having less electrical conductivity than skin away from the bone structure, may affect an effectiveness of the electrodes in measuring biosignals. In addition, as a user wearing the biosensing device moves, the biosensing device itself may shift such that the electrodes may move to less optimal locations for measuring biosignals. Thus, information on electrode placement may be useful for improved measurement accuracy.

The present disclosure is generally directed to electrode placement calibration. As will be explained in greater detail below, embodiments of the present disclosure may detect, in response to driving a reference signal, multiple biosignal measurements using electrodes of a biosensing device and determine, from the biosignal measurements, a relative location of the electrodes with respect to the body to provide feedback based on the relative location. By mapping locations of electrodes with respect to the body, the systems and methods herein may improve biosignal measurement effectiveness.

Features from any of the embodiments described herein may be used in combination with one another in accordance with the general principles described herein. These and other embodiments, features, and advantages will be more fully understood upon reading the following detailed description in conjunction with the accompanying drawings and claims.

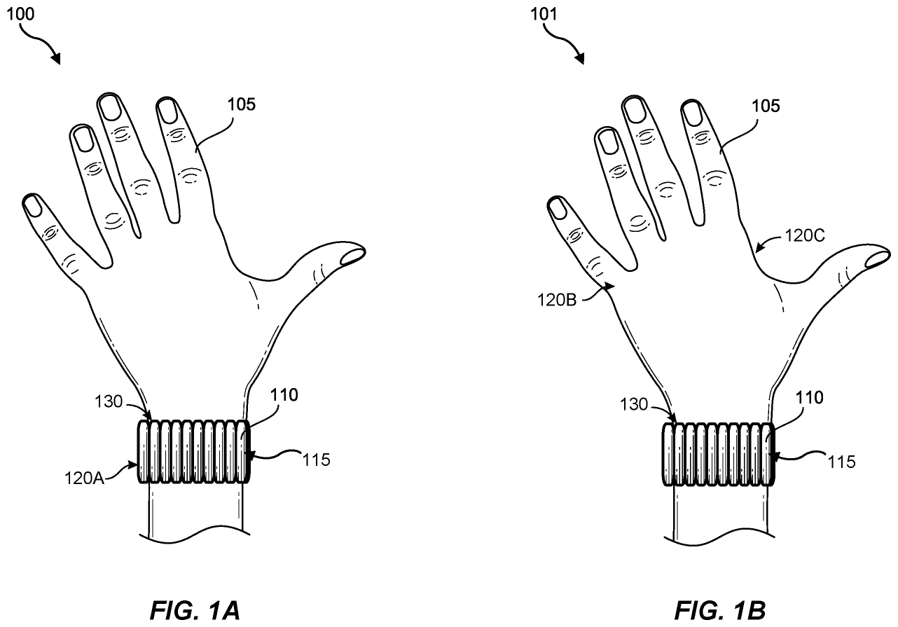

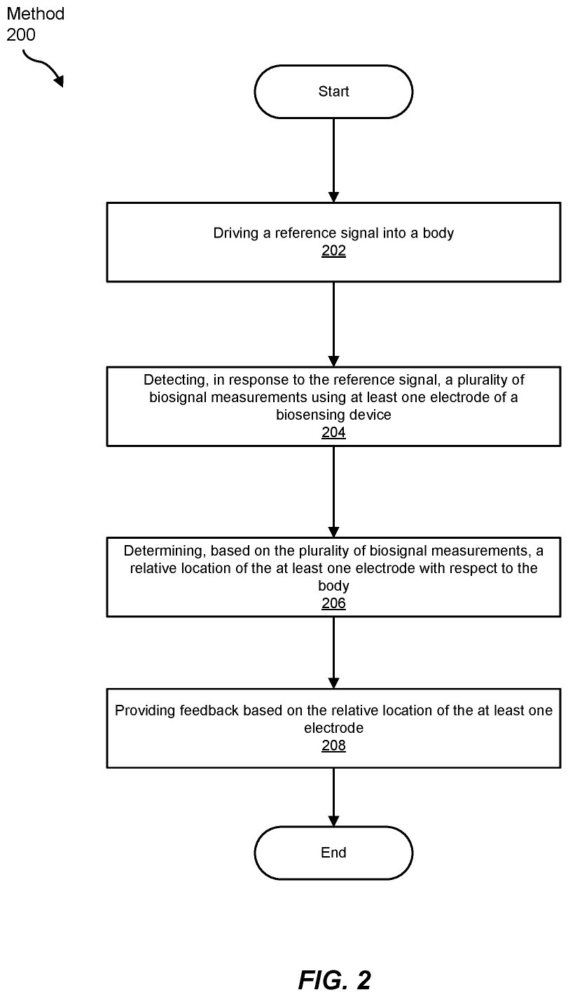

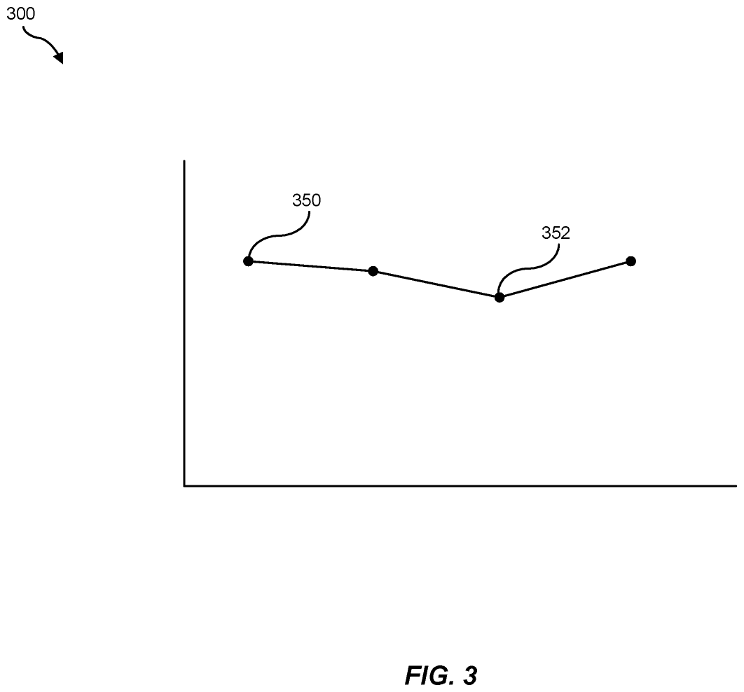

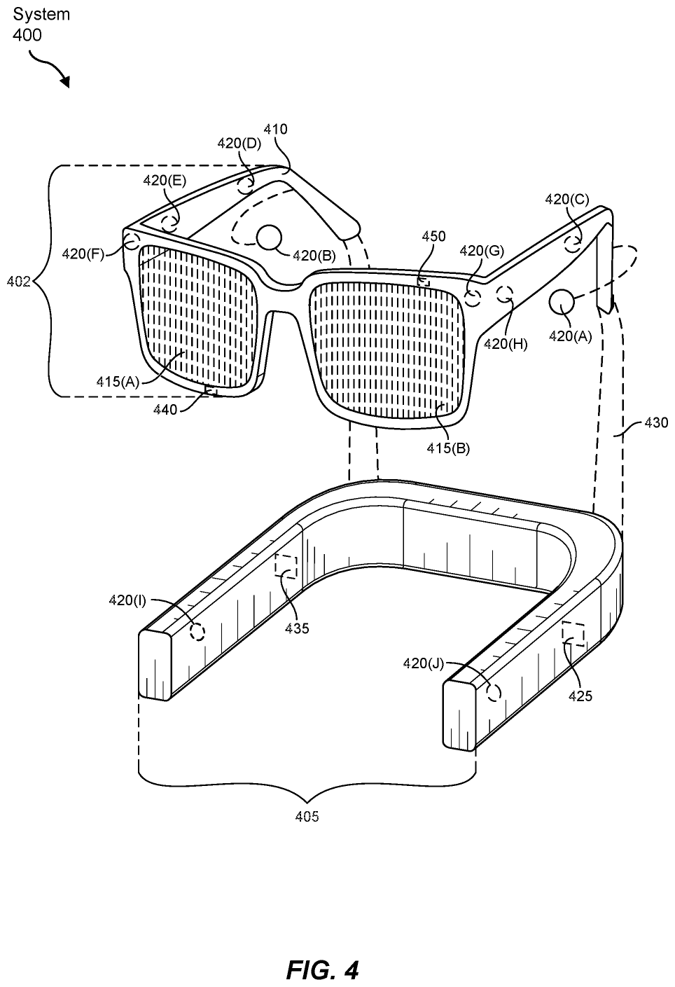

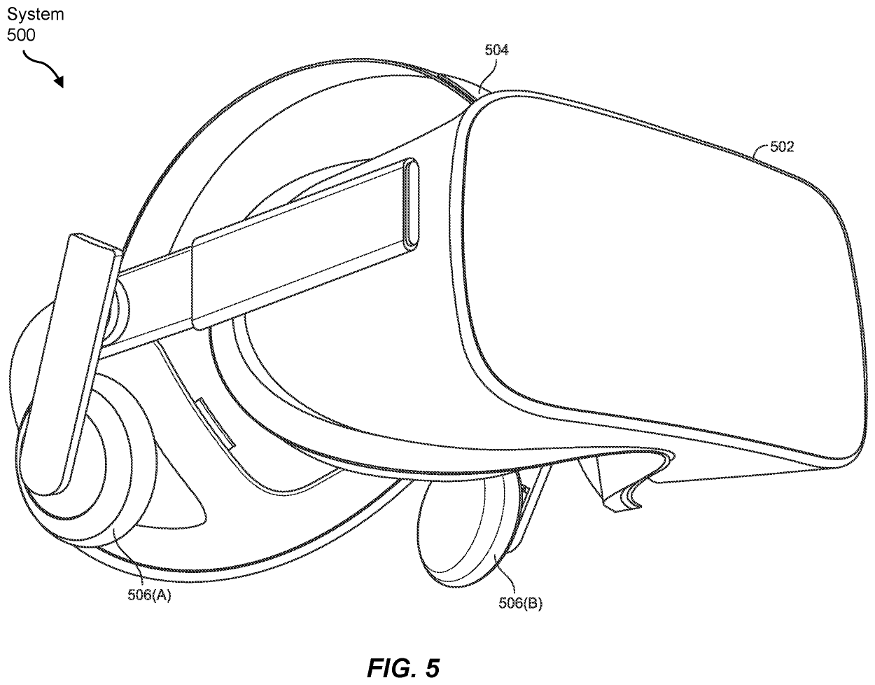

The following will provide, with reference to FIGS. 1-7, detailed descriptions of electrode placement calibration. Detailed descriptions of example systems for electrode placement calibration will be provided in connection with FIGS. 1A, 1B, 6A, 6B, and 7. Detailed descriptions of a method for electrode placement calibration will be provided in connection with FIG. 2 Detailed descriptions of an example signal will be provided in connection with FIG. 3. Detailed descriptions of example related systems will also be provided in connection with FIGS. 4 and 5.

FIGS. 1A and 1B respectively illustrate a biosensing environment 100 and a biosensing environment 101. Biosensing environment 100 and biosensing environment 101 may include a body 105 (e.g., a hand and wrist) and a biosensing device 110. Biosensing device 110 may be a wearable device worn on, for example, a wrist of body 105 and may include an electrode array 115 (not shown) including multiple electrodes contacting body 105 on for example skin at the wrist. Body 105 may have a physiological feature 130 (e.g., a bone such as an ulnar styloid process) that may produce a biosignal minima or maxima when measuring directly at physiological feature 130. For example, in response to a reference stimulus signal, an electrode of electrode array 115 placed onto physiological feature 130 may measure a minima or maxima compared to signals measured elsewhere with electrode array 115. Therefore, physiological feature 130 may be located and used as a reference for mapping the electrodes of electrode array 115 with respect to body 105.

FIG. 1A illustrates an internal stimulus 120A in which biosensing device 110 may drive the reference signal into body 105. Internal stimulus 120A may correspond to an electrical contact integrated with biosensing device 110. In some examples, internal stimulus 120A may correspond to one or more electrodes of electrode array 115.

Ideally, internal stimulus 120A is placed as close to physiological feature 130 as possible. However, even if not ideally placed, the biosignals measured in response to the reference signal may be analyzed to determine the location of physiological feature 130.

FIG. 1B illustrates an external stimulus 120B and an external stimulus 120C which may be separate from, detachable from, and/or extendable from biosensing device 110. Using an external stimulus may allow direct placement at physiological feature 130 or at another physiological feature that may be known to produce an expected signal response. For example, external stimulus 120B may be placed near a metacarpus near the ulnar styloid process (e.g., physiological feature 130). In another example, external stimulus 120C may be placed near a fleshy part of the hand (e.g., the first dorsal interosseous muscle). In yet other examples, the external stimulus may be placed at other physiological features.

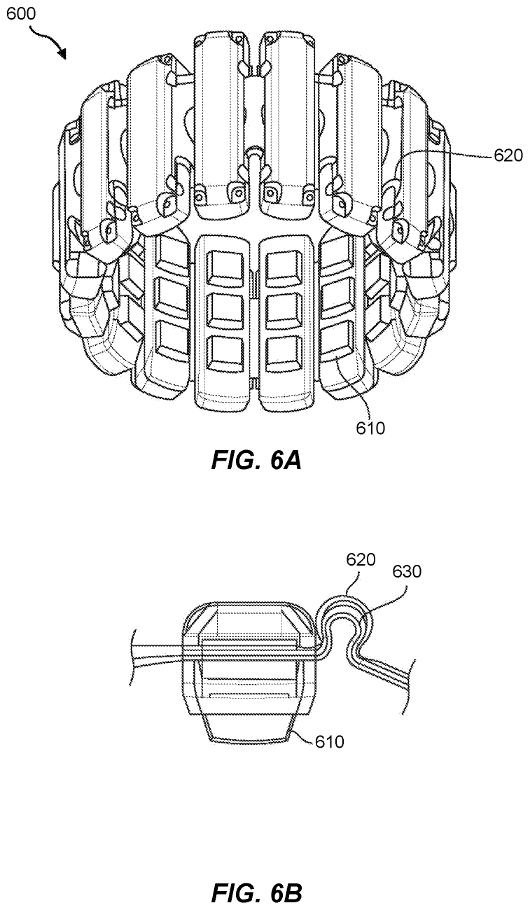

FIG. 2 is a flow diagram of an exemplary computer-implemented method 200 for electrode placement calibration. The steps shown in FIG. 2 may be performed by any suitable computer-executable code and/or computing system, including the system(s) illustrated in FIGS. 1A-1B, 6A-6B, and/or 7A-7B. In one example, each of the steps shown in FIG. 2 may represent an algorithm whose structure includes and/or is represented by multiple sub-steps, examples of which will be provided in greater detail below.

As illustrated in FIG. 2, at step 202 one or more of the systems described herein may drive a reference signal into a body. For example, biosensing device 110 may drive a reference signal into body 105.

In some embodiments, the term “reference signal” may refer to an electrical stimulus signal of known properties to facilitate analysis of a biosignal response to the reference signal. Examples of reference signals include, without limitation, a single tone, a changing waveform, multiple signals, etc. The reference signal may be low intensity so as to be safely driven into a person, such as a signal that is less than 10 μA rms from dc to 1.00 kHz. In other words, the reference signal is generally not noticeable to a person and it complies with the applicable health and safety guidelines for electrical medical devices (e.g., IEC 62368-1 ES1, IEC 60601, and the like).

The systems described herein may perform step 202 in a variety of ways. In one example, driving the reference signal further comprises driving the reference signal using the at least one electrode. For example, as illustrated in FIG. 1A, internal stimulus 120A may be one of the electrodes of electrode array 115. In some examples, driving the reference signal further comprises driving the reference signal using an external stimulus. For example, as illustrated in FIG. 1B external stimulus 120B and/or external stimulus 120C may be used in conjunction with biosensing device 110.

In some examples, one or more of the electrodes of electrode array 115 may be multiplexed such that the multiplexed electrodes may be used for driving one or more signals (e.g., the reference signal and/or any other signal described herein) as well as for measuring biosignals (as will be described further below). In some examples, one or more of the electrodes of electrode array 115 may drive the reference signal (e.g., internal stimulus 120A) while other electrodes of electrode array 115 measure biosignals.

At step 204 one or more of the systems described herein may detect, in response to the reference signal, a plurality of biosignal measurements using at least one electrode of a biosensing device. For example, biosensing device 110 may detect biosignal measurements using electrode array 115.

The systems described herein may perform step 204 in a variety of ways. In one example, the biosensing device comprises a plurality of electrodes and detecting the plurality of biosignal measurements further comprises using the plurality of electrodes to each measure at least one of the plurality of biosignal measurements. For example, each electrode of electrode array 115 may take a biosignal measurement in response to the reference signal.

In other examples, the biosensing device may include a single electrode or a few electrodes. To acquire a sufficient number of biosignal measurements using a limited number of electrodes, in some examples, the reference signal may be driven multiple times, with the biosensing device (e.g., limited electrodes thereof) being shifted by an approximately known amount between each iteration.

In yet other examples (e.g., having multiplexed electrodes in electrode array 115), the electrodes used for driving signals and for measuring signals may shift (e.g., rather than the biosensing device itself being shifted). For instance, a first electrode of electrode array 115 may drive the reference signal (e.g., internal stimulus 120A) while a second electrode of electrode array 115 takes a biosignal measurement in response. On a next iteration, the second electrode (which may be at a different location with respect to body 105 as that of the first electrode) may drive the reference signal while the first electrode (and/or another electrode of electrode array 115) takes a biosignal measurement in response.

At step 206 one or more of the systems described herein may determine, based on the plurality of biosignal measurements, a relative location of the at least one electrode with respect to the body. For example, biosensing device 110 may determine a relative location of one or more of electrode array 115 with respect to body 105.

In some embodiments, the term “relative location” may refer to a location with respect to a particular reference point. For example, in FIGS. 1A-1B, physiological feature 130 may serve as a reference point for describing electrodes of electrode array 115. The relative locations of the electrodes may be defined as linear and/or radial distances from physiological feature 130, orientation with respect to physiological feature 130, and/or other locational relationships to physiological feature 130.

The systems described herein may perform step 206 in a variety of ways. In one example, determining the relative location of the at least one electrode may further include analyzing the plurality of biosignal measurements for a characteristic resultant waveform in response to the reference signal. For example, if the reference signal is a waveform, biosensing device 110 may analyze (using a processor, microprocessor, digital signal processor, machine-learning system, etc.) the biosignal measurements for a waveform, in response to the reference signal, that may provide relative locations of each electrode of electrode array 115 with respect to physiological feature 130.

In some examples, identifying the location of the physiological feature may further include comparing the plurality of biosignal measurements with an expected signal measurement for the reference signal. For example, if biosensing device 110 is expected to be worn on body 105 at a particular location and/or orientation, biosensing device 110 may expect a particular response biosignal measurement in response to the reference signal. A deviation from the expected response may indicate a deviation from the particular location and/or orientation.

In some examples, determining the relative location may further include identifying a location of a physiological feature of the body in response to the reference signal using the plurality of biosignal measurements and determining the relative location of the at least one electrode with respect to the location of the physiological feature. For example, biosensing device 110 may first locate physiological feature 130 (e.g., a location of physiological feature 130 amongst the electrodes of electrode array 115) as a reference point for determining the relative locations of the electrodes of electrode array 115.

FIG. 3 illustrates a simplified graph 300 of biosignals measured by electrodes (represented across an x-axis). For example, FIG. 3 illustrates four biosignal measurements (a strength of which corresponds to a y-axis). Measurement 350 may correspond to a first electrode of an electrode array (e.g., electrode array 115) which may correspond to an internal designation of electrodes. As shown in FIG. 3, measurement 352 may correspond to a minima value, which in some examples, may correspond to a biosignal measured at or near a physiological feature (e.g., physiological feature 130). Thus, the physiological feature may be located at or near an electrode corresponding to measurement 352. Accordingly, the other electrodes may be mapped with respect to a reference location corresponding to the physiological feature.

In some examples, determining the relative location may include multiple iterations of driving the reference signal and taking biosignal measurements. For instance, the biosignal measurements may be averaged, certain values unused (e.g., potential outlier measurements), and/or other statistically analysis may be performed for more accurate measuring. Further, in some examples, the reference signal may be driven from different locations (e.g., using different electrodes) as described herein. In such examples, detecting a similar local minima (e.g., measurement 352) may confirm the location of the physiological feature.

In some examples, determining the relative location may be based on machine learning, statistical analysis, and/or any other heuristic. For example, biosensing device 110 may include and/or otherwise interface with a computing device having a processor and a memory for using machine learning to determine relative locations based on biosignal measurements from the various electrodes of electrode array 115.

Returning to FIG. 2, at step 208 one or more of the systems described herein may provide feedback based on the relative location of the at least one electrode. For example, biosensing device 110 may provide feedback based on the relative location of one or more electrode of electrode array 115.

The systems described herein may perform step 208 in a variety of ways. In one example, biosensing device 110 may further determine an offset from a desired electrode location for the at least one electrode based on the relative location. For example, if the relative location for an electrode is shifted from an expected or desired location (e.g., an expected or desired relative location), biosensing device 110 may determine an offset for how much the electrode is shifted.

In some examples, the feedback may include the offset. For example, biosensing device 110 may provide the offset. In some examples, the feedback may include instructions for repositioning the biosensing device to relocate the electrode from the relative location to the desired electrode location. For example, biosensing device 110 may include instructions to a user (e.g., body 105) wearing biosensing device 110 to reposition biosensing device 110 to relocated electrode array 115 with respect to body 105. The instructions may include, for example, instructions to rotate biosensing device 110 by a certain amount which may correspond to the offset, instructions to flip or reorient biosensing device 110, etc.

In some examples, the feedback may include adjusting signal measurement to account for offset, such as by incorporating the offset with signal measurements when interpreting the signal measurements (e.g., as inputs for analysis such as by a machine learning or other processing). For example, if biosensing device 110 is using the biosignal measurements as user inputs (e.g., detecting gestures), the offset may introduce errors to the user inputs which may have been calibrated for biosensing device 110 worn on body 105 at the desired location and/or orientation. Biosensing device 110 may account for the offset when determining user inputs from the biosignal measurements. In other examples, biosensing device 110 may account for the offset by adjusting biosignal measurement parameters, such as adjusting a gain of affected electrodes (e.g., electrodes that may be near physiological features that may reduce signal measurement performance), adjusting measured values, retraining machine learning models that use biosignal measurements as inputs, etc. By accounting for the offset, the user may not need to reposition biosensing device 110 and biosensing device 110 may be capable of adapting to shifting with respect to body 105.

In some examples, biosensing device 110 may perform the electrode placement calibration described herein (e.g., method 200) periodically. In some examples, biosensing device 110 may perform the electrode placement calibration continuously. In some examples, biosensing device 110 may perform the electrode placement calibration in response to certain triggers, such as a user request, upon powering on, upon detecting one or more errors in biosignal measurements, etc. Moreover, in some examples, biosensing device 110 may timely detect when biosensing device 110 has shifted, to provide feedback as described herein.

In yet other examples, biosensing device 110 may select one or more particular electrodes of electrode array 115 for measuring biosignals. For example, electrodes that are detected as closer to optimal locations may be selected (e.g., via a multiplexer) for measuring biosignals whereas other electrodes, which may be in less optimal locations for measuring biosignals, may be selected for driving signals. Alternatively and/or in addition, certain electrodes, which may be in more optimal locations for driving signals, may be selected for driving signals whereas other electrodes, which may be in less optimal locations for driving signals, may be selected for measuring biosignals. In other words, based on the relative location of electrodes with respect to desired locations (e.g., desired locations for driving signals and/or desired locations for measuring biosignals), biosensing device 110 may designate electrodes of electrode array 115 as signal driving electrodes and/or signal measuring electrodes, including reassigning electrodes as needed. In further examples, the electrodes may be designated as signal measuring electrodes if no signals are to be driven, and the electrodes may be designated as signal driving electrodes if no biosignals are to be measured.

The systems and methods described herein relate to electrode placement calibration. In a many biosensing applications, the electrodes of a biosensing device are expected to be worn in a particular orientation with respect to the body. Thus, wearing the biosensing device incorrectly may yield unexpected or incorrect results.

By using a reference stimulus, either external or internal to the biosensing device, the biosensing device may locate known physiological features with respect to the stimulus location(s). By doing so, the biosensing device may establish a known point of reference for all the biosensing electrodes. The systems and methods described herein allow for a location of an electrode array on the human body due to a reference signal injected into the body.

The reference signal may be integrated into the electrode array or be a separate device. The reference signal may be placed on important physical features such as styloid process radius and a characteristic resultant waveform on the electrodes may be detected. The reference signal may be a single tone or changing waveform that may be analyzed and detected. Downconversion techniques may be used for higher frequency signals that conduct in various ways through the body compared to lower in-band frequencies that an analog front end of a biosensing device is designed for. A user interface or other prompt may allow real time feedback to the user to allow them to locate the reference signal relative to the electrode array.

### Example Embodiments

Example 1: A method comprising: driving a reference signal into a body; detecting, in response to the reference signal, a plurality of biosignal measurements using at least one electrode of a biosensing device; determining, based on the plurality of biosignal measurements, a relative location of the at least one electrode with respect to the body; and providing feedback based on the relative location of the at least one electrode.

Example 2: The method of Example 1, wherein determining the relative location further comprises: identifying a location of a physiological feature of the body in response to the reference signal using the plurality of biosignal measurements; and determining the relative location of the at least one electrode with respect to the location of the physiological feature.

Example 3: The method of Example 2, wherein identifying the location of the physiological feature further comprises comparing the plurality of biosignal measurements with an expected signal measurement for the reference signal.

Example 4: The method of Example 1, 2, or 3, wherein the biosensing device comprises a plurality of electrodes and detecting the plurality of biosignal measurements further comprises using the plurality of electrodes to each measure at least one of the plurality of biosignal measurements.

Example 5: The method of any of Examples 1-4, wherein driving the reference signal further comprises driving the reference signal using the at least one electrode.

Example 6: The method of any of Examples 1-5, wherein driving the reference signal further comprises driving the reference signal using an external stimulus.

Example 7: The method of any of Examples 1-6, wherein the reference signal comprises a single tone or a changing waveform.

Example 8: The method of Example 7, wherein determining the relative location of the at least one electrode further comprises analyzing the plurality of biosignal measurements for a characteristic resultant waveform in response to the reference signal.

Example 9: The method of any of Examples 1-7, further comprising determining an offset from a desired electrode location for the at least one electrode based on the relative location, wherein the feedback includes the offset.

Example 10: The method of Example 9, wherein the feedback includes instructions for repositioning the biosensing device to relocate the electrode from the relative location to the desired electrode location.

Example 11: The method of Example 9 or 10, wherein the feedback includes adjusting signal measurement to account for offset.

Example 12: A biosensing device comprising: at least one electrode for biosignal measurement; at least one physical processor configured to: drive a reference signal into a body; detect, in response to the reference signal, a plurality of biosignal measurements using at least one electrode of a biosensing device; determine, based on the plurality of biosignal measurements, a relative location of the at least one electrode with respect to the body; and provide feedback based on the relative location of the at least one electrode.

Example 13: The biosensing device of Example 12, wherein determining the relative location further comprises: identifying a location of a physiological feature of the body in response to the reference signal using the plurality of biosignal measurements; and determining the relative location of the at least one electrode with respect to the location of the physiological feature.

Example 14: The biosensing device of Example 12 or 13, wherein driving the reference signal further comprises driving the reference signal using the at least one electrode or an external stimulus.

Example 15: The biosensing device of Example 12, 13, or 14, wherein the reference signal comprises a single tone or a changing waveform and determining the relative location of the at least one electrode further comprises analyzing the plurality of biosignal measurements for a characteristic resultant waveform in response to the reference signal.

Example 16: The biosensing device of any of Examples 12-15, wherein the at least one physical processor is further configured to determine an offset from a desired electrode location for the at least one electrode based on the relative location, wherein the feedback includes at least one of: instructions for repositioning the biosensing device to relocate the electrode from the relative location to the desired electrode location, or adjusting signal measurement to account for offset.

Example 17: A system comprising: at least one physical processor; physical memory comprising computer-executable instructions; and a biosensing device comprising: at least one electrode for biosignal measurement; wherein the at least one physical processor is configured to: drive a reference signal into a body; detect, in response to the reference signal, a plurality of biosignal measurements using at least one electrode of a biosensing device; determine, based on the plurality of biosignal measurements, a relative location of the at least one electrode with respect to the body; and provide feedback based on the relative location of the at least one electrode.

Example 18: The system of Example 17, wherein determining the relative location further comprises: identifying a location of a physiological feature of the body in response to the reference signal using the plurality of biosignal measurements; and determining the relative location of the at least one electrode with respect to the location of the physiological feature.

Example 19: The system of Example 17 or 18, further comprising an external stimulus, wherein driving the reference signal further comprises driving the reference signal using the external stimulus.

Example 20: The system of Example 17, 18, or 19, wherein the at least one physical processor is further configured to determine an offset from a desired electrode location for the at least one electrode based on the relative location, wherein the feedback includes at least one of: instructions for repositioning the biosensing device to relocate the electrode from the relative location to the desired electrode location, or adjusting signal measurement to account for offset.

Embodiments of the present disclosure may include or be implemented in conjunction with various types of artificial-reality systems. Artificial reality is a form of reality that has been adjusted in some manner before presentation to a user, which may include, for example, a virtual reality, an augmented reality, a mixed reality, a hybrid reality, or some combination and/or derivative thereof. Artificial-reality content may include completely computer-generated content or computer-generated content combined with captured (e.g., real-world) content. The artificial-reality content may include video, audio, haptic feedback, or some combination thereof, any of which may be presented in a single channel or in multiple channels (such as stereo video that produces a three-dimensional (3D) effect to the viewer). Additionally, in some embodiments, artificial reality may also be associated with applications, products, accessories, services, or some combination thereof, that are used to, for example, create content in an artificial reality and/or are otherwise used in (e.g., to perform activities in) an artificial reality.

Artificial-reality systems may be implemented in a variety of different form factors and configurations. Some artificial-reality systems may be designed to work without near-eye displays (NEDs). Other artificial-reality systems may include an NED that also provides visibility into the real world (such as, e.g., augmented-reality system 400 in FIG. 4) or that visually immerses a user in an artificial reality (such as, e.g., virtual-reality system 500 in FIG. 5). While some artificial-reality devices may be self-contained systems, other artificial-reality devices may communicate and/or coordinate with external devices to provide an artificial-reality experience to a user. Examples of such external devices include handheld controllers, mobile devices, desktop computers, devices worn by a user, devices worn by one or more other users, and/or any other suitable external system.

Turning to FIG. 4, augmented-reality system 400 may include an eyewear device 402 with a frame 410 configured to hold a left display device 415(A) and a right display device 415(B) in front of a user's eyes. Display devices 415(A) and 415(B) may act together or independently to present an image or series of images to a user. While augmented-reality system 400 includes two displays, embodiments of this disclosure may be implemented in augmented-reality systems with a single NED or more than two NEDs.

In some embodiments, augmented-reality system 400 may include one or more sensors, such as sensor 440. Sensor 440 may generate measurement signals in response to motion of augmented-reality system 400 and may be located on substantially any portion of frame 410. Sensor 440 may represent one or more of a variety of different sensing mechanisms, such as a position sensor, an inertial measurement unit (IMU), a depth camera assembly, a structured light emitter and/or detector, or any combination thereof. In some embodiments, augmented-reality system 400 may or may not include sensor 440 or may include more than one sensor. In embodiments in which sensor 440 includes an IMU, the IMU may generate calibration data based on measurement signals from sensor 440. Examples of sensor 440 may include, without limitation, accelerometers, gyroscopes, magnetometers, other suitable types of sensors that detect motion, sensors used for error correction of the IMU, or some combination thereof.

In some examples, augmented-reality system 400 may also include a microphone array with a plurality of acoustic transducers 420(A)-420(J), referred to collectively as acoustic transducers 420. Acoustic transducers 420 may represent transducers that detect air pressure variations induced by sound waves. Each acoustic transducer 420 may be configured to detect sound and convert the detected sound into an electronic format (e.g., an analog or digital format). The microphone array in FIG. 4 may include, for example, ten acoustic transducers: 420(A) and 420(B), which may be designed to be placed inside a corresponding ear of the user, acoustic transducers 420(C), 420(D), 420(E), 420(F), 420(G), and 420(H), which may be positioned at various locations on frame 410, and/or acoustic transducers 420(l) and 420(J), which may be positioned on a corresponding neckband 405.

In some embodiments, one or more of acoustic transducers 420(A)-(J) may be used as output transducers (e.g., speakers). For example, acoustic transducers 420(A) and/or 420(B) may be earbuds or any other suitable type of headphone or speaker.

The configuration of acoustic transducers 420 of the microphone array may vary. While augmented-reality system 400 is shown in FIG. 4 as having ten acoustic transducers 420, the number of acoustic transducers 420 may be greater or less than ten. In some embodiments, using higher numbers of acoustic transducers 420 may increase the amount of audio information collected and/or the sensitivity and accuracy of the audio information. In contrast, using a lower number of acoustic transducers 420 may decrease the computing power required by an associated controller 450 to process the collected audio information. In addition, the position of each acoustic transducer 420 of the microphone array may vary. For example, the position of an acoustic transducer 420 may include a defined position on the user, a defined coordinate on frame 410, an orientation associated with each acoustic transducer 420, or some combination thereof.

Acoustic transducers 420(A) and 420(B) may be positioned on different parts of the user's ear, such as behind the pinna, behind the tragus, and/or within the auricle or fossa. Or, there may be additional acoustic transducers 420 on or surrounding the ear in addition to acoustic transducers 420 inside the ear canal. Having an acoustic transducer 420 positioned next to an ear canal of a user may enable the microphone array to collect information on how sounds arrive at the ear canal. By positioning at least two of acoustic transducers 420 on either side of a user's head (e.g., as binaural microphones), augmented-reality system 400 may simulate binaural hearing and capture a 3D stereo sound field around about a user's head. In some embodiments, acoustic transducers 420(A) and 420(B) may be connected to augmented-reality system 400 via a wired connection 430, and in other embodiments acoustic transducers 420(A) and 420(B) may be connected to augmented-reality system 400 via a wireless connection (e.g., a BLUETOOTH connection). In still other embodiments, acoustic transducers 420(A) and 420(B) may not be used at all in conjunction with augmented-reality system 400.

Acoustic transducers 420 on frame 410 may be positioned in a variety of different ways, including along the length of the temples, across the bridge, above or below display devices 415(A) and 415(B), or some combination thereof. Acoustic transducers 420 may also be oriented such that the microphone array is able to detect sounds in a wide range of directions surrounding the user wearing the augmented-reality system 400. In some embodiments, an optimization process may be performed during manufacturing of augmented-reality system 400 to determine relative positioning of each acoustic transducer 420 in the microphone array.

In some examples, augmented-reality system 400 may include or be connected to an external device (e.g., a paired device), such as neckband 405. Neckband 405 generally represents any type or form of paired device. Thus, the following discussion of neckband 405 may also apply to various other paired devices, such as charging cases, smart watches, smart phones, wrist bands, other wearable devices, hand-held controllers, tablet computers, laptop computers, other external compute devices, etc.

As shown, neckband 405 may be coupled to eyewear device 402 via one or more connectors. The connectors may be wired or wireless and may include electrical and/or non-electrical (e.g., structural) components. In some cases, eyewear device 402 and neckband 405 may operate independently without any wired or wireless connection between them. While FIG. 4 illustrates the components of eyewear device 402 and neckband 405 in example locations on eyewear device 402 and neckband 405, the components may be located elsewhere and/or distributed differently on eyewear device 402 and/or neckband 405. In some embodiments, the components of eyewear device 402 and neckband 405 may be located on one or more additional peripheral devices paired with eyewear device 402, neckband 405, or some combination thereof.

Pairing external devices, such as neckband 405, with augmented-reality eyewear devices may enable the eyewear devices to achieve the form factor of a pair of glasses while still providing sufficient battery and computation power for expanded capabilities. Some or all of the battery power, computational resources, and/or additional features of augmented-reality system 400 may be provided by a paired device or shared between a paired device and an eyewear device, thus reducing the weight, heat profile, and form factor of the eyewear device overall while still retaining desired functionality. For example, neckband 405 may allow components that would otherwise be included on an eyewear device to be included in neckband 405 since users may tolerate a heavier weight load on their shoulders than they would tolerate on their heads. Neckband 405 may also have a larger surface area over which to diffuse and disperse heat to the ambient environment. Thus, neckband 405 may allow for greater battery and computation capacity than might otherwise have been possible on a stand-alone eyewear device. Since weight carried in neckband 405 may be less invasive to a user than weight carried in eyewear device 402, a user may tolerate wearing a lighter eyewear device and carrying or wearing the paired device for greater lengths of time than a user would tolerate wearing a heavy standalone eyewear device, thereby enabling users to more fully incorporate artificial-reality environments into their day-to-day activities.

Neckband 405 may be communicatively coupled with eyewear device 402 and/or to other devices. These other devices may provide certain functions (e.g., tracking, localizing, depth mapping, processing, storage, etc.) to augmented-reality system 400. In the embodiment of FIG. 4, neckband 405 may include two acoustic transducers (e.g., 420(l) and 420(J)) that are part of the microphone array (or potentially form their own microphone subarray). Neckband 405 may also include a controller 425 and a power source 435.

Acoustic transducers 420(l) and 420(J) of neckband 405 may be configured to detect sound and convert the detected sound into an electronic format (analog or digital). In the embodiment of FIG. 4, acoustic transducers 420(l) and 420(J) may be positioned on neckband 405, thereby increasing the distance between the neckband acoustic transducers 420(l) and 420(J) and other acoustic transducers 420 positioned on eyewear device 402. In some cases, increasing the distance between acoustic transducers 420 of the microphone array may improve the accuracy of beamforming performed via the microphone array. For example, if a sound is detected by acoustic transducers 420(C) and 420(D) and the distance between acoustic transducers 420(C) and 420(D) is greater than, e.g., the distance between acoustic transducers 420(D) and 420(E), the determined source location of the detected sound may be more accurate than if the sound had been detected by acoustic transducers 420(D) and 420(E).

Controller 425 of neckband 405 may process information generated by the sensors on neckband 405 and/or augmented-reality system 400. For example, controller 425 may process information from the microphone array that describes sounds detected by the microphone array. For each detected sound, controller 425 may perform a direction-of-arrival (DOA) estimation to estimate a direction from which the detected sound arrived at the microphone array. As the microphone array detects sounds, controller 425 may populate an audio data set with the information. In embodiments in which augmented-reality system 400 includes an inertial measurement unit, controller 425 may compute all inertial and spatial calculations from the IMU located on eyewear device 402. A connector may convey information between augmented-reality system 400 and neckband 405 and between augmented-reality system 400 and controller 425. The information may be in the form of optical data, electrical data, wireless data, or any other transmittable data form. Moving the processing of information generated by augmented-reality system 400 to neckband 405 may reduce weight and heat in eyewear device 402, making it more comfortable to the user.

Power source 435 in neckband 405 may provide power to eyewear device 402 and/or to neckband 405. Power source 435 may include, without limitation, lithium ion batteries, lithium-polymer batteries, primary lithium batteries, alkaline batteries, or any other form of power storage. In some cases, power source 435 may be a wired power source. Including power source 435 on neckband 405 instead of on eyewear device 402 may help better distribute the weight and heat generated by power source 435.

As noted, some artificial-reality systems may, instead of blending an artificial reality with actual reality, substantially replace one or more of a user's sensory perceptions of the real world with a virtual experience. One example of this type of system is a head-worn display system, such as virtual-reality system 500 in FIG. 5, that mostly or completely covers a user's field of view. Virtual-reality system 500 may include a front rigid body 502 and a band 504 shaped to fit around a user's head. Virtual-reality system 500 may also include output audio transducers 506(A) and 506(B). Furthermore, while not shown in FIG. 5, front rigid body 502 may include one or more electronic elements, including one or more electronic displays, one or more inertial measurement units (IMUs), one or more tracking emitters or detectors, and/or any other suitable device or system for creating an artificial-reality experience.

Artificial-reality systems may include a variety of types of visual feedback mechanisms. For example, display devices in augmented-reality system 400 and/or virtual-reality system 500 may include one or more liquid crystal displays (LCDs), light emitting diode (LED) displays, microLED displays, organic LED (OLED) displays, digital light project (DLP) micro-displays, liquid crystal on silicon (LCoS) micro-displays, and/or any other suitable type of display screen. These artificial-reality systems may include a single display screen for both eyes or may provide a display screen for each eye, which may allow for additional flexibility for varifocal adjustments or for correcting a user's refractive error. Some of these artificial-reality systems may also include optical subsystems having one or more lenses (e.g., concave or convex lenses, Fresnel lenses, adjustable liquid lenses, etc.) through which a user may view a display screen. These optical subsystems may serve a variety of purposes, including to collimate (e.g., make an object appear at a greater distance than its physical distance), to magnify (e.g., make an object appear larger than its actual size), and/or to relay (to, e.g., the viewer's eyes) light. These optical subsystems may be used in a non-pupil-forming architecture (such as a single lens configuration that directly collimates light but results in so-called pincushion distortion) and/or a pupil-forming architecture (such as a multi-lens configuration that produces so-called barrel distortion to nullify pincushion distortion).

In addition to or instead of using display screens, some of the artificial-reality systems described herein may include one or more projection systems. For example, display devices in augmented-reality system 400 and/or virtual-reality system 500 may include microLED projectors that project light (using, e.g., a waveguide) into display devices, such as clear combiner lenses that allow ambient light to pass through. The display devices may refract the projected light toward a user's pupil and may enable a user to simultaneously view both artificial-reality content and the real world. The display devices may accomplish this using any of a variety of different optical components, including waveguide components (e.g., holographic, planar, diffractive, polarized, and/or reflective waveguide elements), light-manipulation surfaces and elements (such as diffractive, reflective, and refractive elements and gratings), coupling elements, etc. Artificial-reality systems may also be configured with any other suitable type or form of image projection system, such as retinal projectors used in virtual retina displays.

The artificial-reality systems described herein may also include various types of computer vision components and subsystems. For example, augmented-reality system 400 and/or virtual-reality system 500 may include one or more optical sensors, such as two-dimensional (2D) or 3D cameras, structured light transmitters and detectors, time-of-flight depth sensors, single-beam or sweeping laser rangefinders, 3D LiDAR sensors, and/or any other suitable type or form of optical sensor. An artificial-reality system may process data from one or more of these sensors to identify a location of a user, to map the real world, to provide a user with context about real-world surroundings, and/or to perform a variety of other functions.

The artificial-reality systems described herein may also include one or more input and/or output audio transducers. Output audio transducers may include voice coil speakers, ribbon speakers, electrostatic speakers, piezoelectric speakers, bone conduction transducers, cartilage conduction transducers, tragus-vibration transducers, and/or any other suitable type or form of audio transducer. Similarly, input audio transducers may include condenser microphones, dynamic microphones, ribbon microphones, and/or any other type or form of input transducer. In some embodiments, a single transducer may be used for both audio input and audio output.

In some embodiments, the artificial-reality systems described herein may also include tactile (i.e., haptic) feedback systems, which may be incorporated into headwear, gloves, body suits, handheld controllers, environmental devices (e.g., chairs, floormats, etc.), and/or any other type of device or system. Haptic feedback systems may provide various types of cutaneous feedback, including vibration, force, traction, texture, and/or temperature. Haptic feedback systems may also provide various types of kinesthetic feedback, such as motion and compliance. Haptic feedback may be implemented using motors, piezoelectric actuators, fluidic systems, and/or a variety of other types of feedback mechanisms. Haptic feedback systems may be implemented independent of other artificial-reality devices, within other artificial-reality devices, and/or in conjunction with other artificial-reality devices.

By providing haptic sensations, audible content, and/or visual content, artificial-reality systems may create an entire virtual experience or enhance a user's real-world experience in a variety of contexts and environments. For instance, artificial-reality systems may assist or extend a user's perception, memory, or cognition within a particular environment. Some systems may enhance a user's interactions with other people in the real world or may enable more immersive interactions with other people in a virtual world. Artificial-reality systems may also be used for educational purposes (e.g., for teaching or training in schools, hospitals, government organizations, military organizations, business enterprises, etc.), entertainment purposes (e.g., for playing video games, listening to music, watching video content, etc.), and/or for accessibility purposes (e.g., as hearing aids, visual aids, etc.). The embodiments disclosed herein may enable or enhance a user's artificial-reality experience in one or more of these contexts and environments and/or in other contexts and environments.

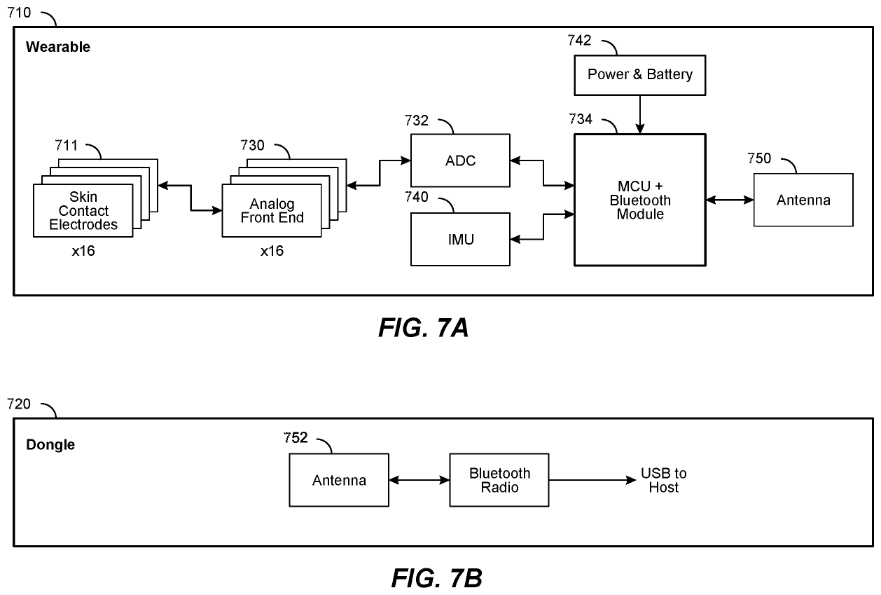

FIG. 6A illustrates an exemplary human-machine interface (also referred to herein as an EMG control interface) configured to be worn around a user's lower arm or wrist as a wearable system 600. In this example, wearable system 600 may include sixteen neuromuscular sensors 610 (e.g., EMG sensors) arranged circumferentially around an elastic band 620 with an interior surface 630 configured to contact a user's skin. However, any suitable number of neuromuscular sensors may be used. The number and arrangement of neuromuscular sensors may depend on the particular application for which the wearable device is used. For example, a wearable armband or wristband can be used to generate control information for controlling an augmented reality system, a robot, controlling a vehicle, scrolling through text, controlling a virtual avatar, or any other suitable control task. As shown, the sensors may be coupled together using flexible electronics incorporated into the wireless device. FIG. 6B illustrates a cross-sectional view through one of the sensors of the wearable device shown in FIG. 6A. In some embodiments, the output of one or more of the sensing components can be optionally processed using hardware signal processing circuitry (e.g., to perform amplification, filtering, and/or rectification). In other embodiments, at least some signal processing of the output of the sensing components can be performed in software. Thus, signal processing of signals sampled by the sensors can be performed in hardware, software, or by any suitable combination of hardware and software, as aspects of the technology described herein are not limited in this respect. A non-limiting example of a signal processing chain used to process recorded data from sensors 610 is discussed in more detail below with reference to FIGS. 7A and 7B.

FIGS. 7A and 7B illustrate an exemplary schematic diagram with internal components of a wearable system with EMG sensors. As shown, the wearable system may include a wearable portion 710 (FIG. 7A) and a dongle portion 720 (FIG. 7B) in communication with the wearable portion 710 (e.g., via BLUETOOTH or another suitable wireless communication technology). As shown in FIG. 7A, the wearable portion 710 may include skin contact electrodes 711, examples of which are described in connection with FIGS. 6A and 6B. The output of the skin contact electrodes 711 may be provided to analog front end 730, which may be configured to perform analog processing (e.g., amplification, noise reduction, filtering, etc.) on the recorded signals. The processed analog signals may then be provided to analog-to-digital converter 732, which may convert the analog signals to digital signals that can be processed by one or more computer processors. An example of a computer processor that may be used in accordance with some embodiments is microcontroller (MCU) 734, illustrated in FIG. 7A. As shown, MCU 734 may also include inputs from other sensors (e.g., IMU sensor 740), and power and battery module 742. The output of the processing performed by MCU 734 may be provided to antenna 750 for transmission to dongle portion 720 shown in FIG. 7B.

Dongle portion 720 may include antenna 752, which may be configured to communicate with antenna 750 included as part of wearable portion 710. Communication between antennas 750 and 752 may occur using any suitable wireless technology and protocol, non-limiting examples of which include radiofrequency signaling and BLUETOOTH. As shown, the signals received by antenna 752 of dongle portion 720 may be provided to a host computer for further processing, display, and/or for effecting control of a particular physical or virtual object or objects.

Although the examples provided with reference to FIGS. 6A-6B and FIGS. 7A-7B are discussed in the context of interfaces with EMG sensors, the techniques described herein for reducing electromagnetic interference can also be implemented in wearable interfaces with other types of sensors including, but not limited to, mechanomyography (MMG) sensors, sonomyography (SMG) sensors, and electrical impedance tomography (EIT) sensors. The techniques described herein for reducing electromagnetic interference can also be implemented in wearable interfaces that communicate with computer hosts through wires and cables (e.g., USB cables, optical fiber cables, etc.).

In some examples, the term “memory device” generally refers to any type or form of volatile or non-volatile storage device or medium capable of storing data and/or computer-readable instructions. In one example, a memory device may store, load, and/or maintain one or more of the modules described herein. Examples of memory devices include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In some examples, the term “physical processor” generally refers to any type or form of hardware-implemented processing unit capable of interpreting and/or executing computer-readable instructions. In one example, a physical processor may access and/or modify one or more modules stored in the above-described memory device. Examples of physical processors include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

In some embodiments, the term “computer-readable medium” generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A method comprising:
driving a reference signal into a body;
detecting, in response to the reference signal, a plurality of biosignal measurements using at least one electrode of a biosensing device;
determining, based on the plurality of biosignal measurements, a relative location of the at least one electrode with respect to the body; and
providing feedback based on the relative location of the at least one electrode.

2. The method of claim 1, wherein determining the relative location further comprises:
identifying a location of a physiological feature of the body in response to the reference signal using the plurality of biosignal measurements; and
determining the relative location of the at least one electrode with respect to the location of the physiological feature.

3. The method of claim 2, wherein identifying the location of the physiological feature further comprises comparing the plurality of biosignal measurements with an expected signal measurement for the reference signal.

4. The method of claim 1, wherein the biosensing device comprises a plurality of electrodes and detecting the plurality of biosignal measurements further comprises using the plurality of electrodes to each measure at least one of the plurality of biosignal measurements.

5. The method of claim 1, wherein driving the reference signal further comprises driving the reference signal using the at least one electrode.

6. The method of claim 1, wherein driving the reference signal further comprises driving the reference signal using an external stimulus.

7. The method of claim 1, wherein the reference signal comprises a single tone or a changing waveform.

8. The method of claim 7, wherein determining the relative location of the at least one electrode further comprises analyzing the plurality of biosignal measurements for a characteristic resultant waveform in response to the reference signal.

9. The method of claim 1, further comprising determining an offset from a desired electrode location for the at least one electrode based on the relative location, wherein the feedback includes the offset.

10. The method of claim 9, wherein the feedback includes instructions for repositioning the biosensing device to relocate the electrode from the relative location to the desired electrode location.

11. The method of claim 9, wherein the feedback includes adjusting signal measurement to account for offset.

12. A biosensing device comprising:
at least one electrode for biosignal measurement;
at least one physical processor configured to:
drive a reference signal into a body;
detect, in response to the reference signal, a plurality of biosignal measurements using at least one electrode of a biosensing device;
determine, based on the plurality of biosignal measurements, a relative location of the at least one electrode with respect to the body; and
provide feedback based on the relative location of the at least one electrode.

13. The biosensing device of claim 12, wherein determining the relative location further comprises:
identifying a location of a physiological feature of the body in response to the reference signal using the plurality of biosignal measurements; and
determining the relative location of the at least one electrode with respect to the location of the physiological feature.

14. The biosensing device of claim 12, wherein driving the reference signal further comprises driving the reference signal using the at least one electrode or an external stimulus.

15. The biosensing device of claim 12, wherein the reference signal comprises a single tone or a changing waveform and determining the relative location of the at least one electrode further comprises analyzing the plurality of biosignal measurements for a characteristic resultant waveform in response to the reference signal.

16. The biosensing device of claim 12, wherein the at least one physical processor is further configured to determine an offset from a desired electrode location for the at least one electrode based on the relative location, wherein the feedback includes at least one of: instructions for repositioning the biosensing device to relocate the electrode from the relative location to the desired electrode location, or adjusting signal measurement to account for offset.

17. A system comprising:
at least one physical processor;
physical memory comprising computer-executable instructions; and
a biosensing device comprising:
at least one electrode for biosignal measurement;
wherein the at least one physical processor is configured to:
drive a reference signal into a body;
detect, in response to the reference signal, a plurality of biosignal measurements using at least one electrode of a biosensing device;
determine, based on the plurality of biosignal measurements, a relative location of the at least one electrode with respect to the body; and
provide feedback based on the relative location of the at least one electrode.

18. The system of claim 17, wherein determining the relative location further comprises:
identifying a location of a physiological feature of the body in response to the reference signal using the plurality of biosignal measurements; and
determining the relative location of the at least one electrode with respect to the location of the physiological feature.

19. The system of claim 17, further comprising an external stimulus, wherein driving the reference signal further comprises driving the reference signal using the external stimulus.

20. The system of claim 17, wherein the at least one physical processor is further configured to determine an offset from a desired electrode location for the at least one electrode based on the relative location, wherein the feedback includes at least one of: instructions for repositioning the biosensing device to relocate the electrode from the relative location to the desired electrode location, or adjusting signal measurement to account for offset.
